(19) United States
(12) Patent Application Publication
(10) Pub. No.: US 2026/0363664 A1
(43) Pub. Date: Nov. 19, 2026

(54) NONLINEAR LAYER TO AUGMENT SOUND PROPAGATION AND IMAGING

(71) Applicant: Georgia Tech Research Corporation, Atlanta, GA (US)

(72) Inventors: Konstantinos Arvanitis, Atlanta, GA (US); Pradosh Pritam Dash, Atlanta, GA (US)

(21) Appl. No.: 19/679,527

(22) Filed: May 15, 2026

Related U.S. Application Data
(60) Provisional application No. 63/806,113, filed on May 15, 2025.

(51) Int. Cl. A61B 8/00; A61B 8/08; G01S 7/521; G10K 11/30

(52) U.S. Cl. CPC A61B 8/4494; A61B 8/0808; A61B 8/5207; A61B 8/54; G01S 7/521; G10K 11/30

(57) ABSTRACT

The present disclosure relates generally to nonlinear acoustic wave propagation and ultrasound systems, and particularly to focused ultrasound therapy registration. A system includes an ultrasound transducer configured to generate a first acoustic wave at a first frequency and a second acoustic wave at a second frequency, a holographic lens coupled to the transducer and configured to focus acoustic energy toward a target region through a nonlinear medium, a receiver configured to detect a parametric array signal generated by nonlinear mixing of the first and second acoustic waves within the nonlinear medium, wherein the parametric array signal includes a difference frequency component corresponding to a difference between the first and second frequencies, and a controller configured to receive the parametric array signal from the receiver and determine alignment status between the holographic lens and the nonlinear medium based on a characteristic of the parametric array signal.

### CROSS-REFERENCE TO RELATED APPLICATIONS

This application claims the benefit of U.S. Provisional Application Ser. No. 63/806,113, filed on 15 May 2025, which is incorporated herein by reference in its entirety as if fully set forth below.

### GOVERNMENT LICENSE RIGHTS

This invention was made with government support under 1933158, and 1966158 awarded by the National Science Foundation. The government has certain rights in this invention.

### FIELD OF INVENTION

The present disclosure relates to nonlinear acoustic wave propagation and ultrasound systems, and more particularly to systems and methods utilizing nonlinear layers to augment parametric array signal generation for enhanced harmonic imaging and focused ultrasound therapy registration.

### BACKGROUND

Nonlinear acoustics have emerged as a promising field with potential across various applications, particularly in biomedical ultrasonics. In this domain, harmonic imaging can enhance ultrasound images by providing improved contrast, which may be valuable for accurate diagnostics and therapeutic interventions. Ultrasound systems may utilize nonlinear wave propagation effects, including the parametric array effect, where two high-frequency sound beams of finite amplitude interact to produce sum and difference frequency beams. The strength of these difference frequency signals, often termed parametric array signals, possesses properties including high directionality and the ability to penetrate through structures such as the skull, making them potentially valuable for focused ultrasound therapy and imaging applications.

In transcranial focused ultrasound applications, holographic lenses or phase plates may be employed to correct for aberrations introduced by the skull and achieve focused acoustic energy delivery to target regions within the brain. These holographic lenses are typically designed based on patient-specific skull geometry obtained from imaging data. However, achieving accurate registration between the holographic lens and the patient's skull during treatment remains a challenge. Misregistration between the lens and the skull can introduce aberrations that degrade the quality of the focused ultrasound beam, potentially reducing therapeutic efficacy or affecting imaging quality. Current approaches for registration may rely on external tracking systems or imaging guidance, which can add complexity to the treatment workflow.

Additionally, existing ultrasound systems may not fully exploit the nonlinear properties of intervening structures or tissues to enhance signal generation. While impedance matching layers are used extensively in ultrasonics to match the acoustic impedance of the transducer with that of the imaging subject to maximize ultrasound transmission, approaches for augmenting nonlinear wave propagation through engineered layers remain limited. The generation of harmonics, self-phase demodulation, and other nonlinear acoustic effects typically occurs over extended propagation distances, which may limit their utility in certain applications where shorter interaction lengths are desirable.

What is needed, therefore, are systems and methods that can utilize nonlinear layers to augment parametric array signal generation for enhanced harmonic imaging and focused ultrasound therapy registration. Such systems and methods may provide real-time feedback mechanisms for noninvasive alignment of holographic lenses with patient anatomy, while also supporting the development of high-sensitivity harmonic imaging, shock wave formation, and therapeutic interventions that employ nonlinear acoustics.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

A system for focused ultrasound therapy registration can include an ultrasound transducer configured to generate a first acoustic wave at a first frequency and a second acoustic wave at a second frequency. The system can include a holographic lens coupled to the ultrasound transducer and configured to focus acoustic energy toward a target region through a nonlinear medium. The system can include a receiver configured to detect a parametric array signal generated by nonlinear mixing of the first acoustic wave and the second acoustic wave within the nonlinear medium, wherein the parametric array signal includes a difference frequency component corresponding to a difference between the first frequency and the second frequency. The system can include a controller configured to receive the parametric array signal from the receiver and determine an alignment status between the holographic lens and the nonlinear medium based on a characteristic of the parametric array signal.

A method for registering a holographic lens with a nonlinear medium can include transmitting, from an ultrasound transducer coupled to the holographic lens, a first acoustic wave at a first frequency and a second acoustic wave at a second frequency toward the nonlinear medium. The method can include detecting, with a receiver, a parametric array signal generated by nonlinear mixing of the first acoustic wave and the second acoustic wave within the nonlinear medium, wherein the parametric array signal includes a difference frequency component corresponding to a difference between the first frequency and the second frequency. The method can include analyzing the parametric array signal to determine an alignment status between the holographic lens and the nonlinear medium. The method can include adjusting a position or orientation of the holographic lens relative to the nonlinear medium based on the alignment status.

A system for enhancing nonlinear acoustic wave propagation can include an ultrasound transducer configured to generate a first acoustic wave at a first frequency and a second acoustic wave at a second frequency. The system can include a nonlinear layer positioned in a propagation path of the first acoustic wave and the second acoustic wave, wherein the nonlinear layer is configured to augment generation of a parametric array signal through nonlinear mixing of the first acoustic wave and the second acoustic wave. The system can include a receiver configured to detect the parametric array signal, wherein the parametric array signal includes a difference frequency component corresponding to a difference between the first frequency and the second frequency.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a system for focused ultrasound therapy registration, according to examples of the disclosed technology.

FIG. 2A illustrates a frequency domain representation of a source pressure signal, according to examples of the disclosed technology.

FIG. 2B illustrates a frequency domain representation of a received signal with a difference frequency component, according to examples of the disclosed technology.

FIG. 3 illustrates nonlinear mixing of acoustic waves through a skull, according to examples of the disclosed technology.

FIG. 4 illustrates a relationship between parametric array signal strength and misregistration, according to examples of the disclosed technology.

FIG. 5 illustrates nonlinear sound propagation and parametric array signal generation through a skull, according to examples of the disclosed technology.

FIG. 6 illustrates a comparison of parametric array signal generation with and without skull corrections, according to examples of the disclosed technology.

FIG. 7 illustrates a relationship between normalized level and misregistration angle, according to examples of the disclosed technology.

FIG. 8A illustrates pressure measurements as a function of position for a primary frequency, according to examples of the disclosed technology.

FIG. 8B illustrates pressure measurements as a function of position for a difference frequency, according to examples of the disclosed technology.

FIG. 9 illustrates a relationship between parametric pressure drop and rotational angle for different skull nonlinearity values, according to examples of the disclosed technology.

FIG. 10 illustrates variation of parametric pressure with rotational angle for different axes of rotation, according to examples of the disclosed technology.

FIG. 11 illustrates an effect of varying difference frequencies on parametric pressure drop, according to examples of the disclosed technology.

FIG. 12 illustrates a relationship between F-number and parametric pressure drop sensitivity, according to examples of the disclosed technology.

FIG. 13A illustrates an input signal in time and frequency domains, according to examples of the disclosed technology.

FIG. 13B illustrates a measured signal with nonlinear distortion in time and frequency domains, according to examples of the disclosed technology.

FIG. 14 illustrates progressive nonlinear distortion of a measured signal at different axial positions, according to examples of the disclosed technology.

FIG. 15 illustrates an axial line scan showing normalized amplitude versus axial position, according to examples of the disclosed technology.

FIG. 16 illustrates simulation results showing a relationship between normalized level and rotation angle, according to examples of the disclosed technology.

FIG. 17 illustrates experimental variation of parametric pressure as a function of rotation angle, according to examples of the disclosed technology.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The disclosed technology relates to nonlinear acoustic wave propagation and ultrasound systems. In some cases, the disclosed technology can be applied to focused ultrasound therapy registration, harmonic imaging, and therapeutic interventions that employ nonlinear acoustics.

Nonlinear acoustic effects can arise when acoustic waves of finite amplitude propagate through a medium. The parametric array effect is one such nonlinear wave propagation effect, where two high-frequency sound beams interact to produce sum and difference frequency beams. The parametric array signal, which comprises the difference frequency component, possesses properties including high directionality and the ability to penetrate through structures such as bone tissue. The strength of the parametric array signal can be proportional to the nonlinearity parameter of the medium, the amplitude of the primary beams, and the propagation length.

In transcranial focused ultrasound applications, holographic lenses or phase plates can be employed to correct for aberrations introduced by the skull and achieve focused acoustic energy delivery to target regions within the brain. A holographic lens can be designed based on patient-specific skull geometry obtained from imaging data. Achieving accurate registration between the holographic lens and the patient's skull during treatment can affect the quality of the focused ultrasound beam. Misregistration between the lens and the skull can introduce aberrations that affect the focal characteristics of the acoustic energy.

The disclosed technology can utilize nonlinear layers to augment parametric array signal generation. A nonlinear layer positioned in the propagation path of acoustic waves can enhance the generation of harmonics, self-phase demodulation, and other nonlinear acoustic effects. The nonlinear layer can be configured to augment generation of the parametric array signal by virtue of having a nonlinearity parameter substantially greater than surrounding media, such as water or soft tissue, which enhances the nonlinear mixing of the acoustic waves. In some cases, a nonlinear layer can support the development of finite amplitude effects at shorter propagation distances than would occur in the absence of the nonlinear layer. With sufficiently high nonlinearity at the start of the propagation path, the difference frequency signal can be generated within confined spaces or at very short propagation distances where conventional nonlinear signal generation would not be possible. This is because a highly nonlinear layer can generate the parametric array signal without requiring extended cumulative propagation distances that are typically needed for nonlinear effects to develop. The nonlinear layer can be analogous to impedance matching layers used in ultrasonics, which match the acoustic impedance of the transducer with that of the imaging subject to maximize ultrasound transmission.

In some cases, the disclosed technology can provide a feedback mechanism for noninvasive alignment of holographic lenses with patient anatomy. The parametric array signal can serve as a registration metric, where changes in the parametric array signal correspond to the alignment status between the holographic lens and a nonlinear medium such as the skull. The relationship between skull nonlinearity and aberrations caused by misregistration can be utilized to determine when the holographic lens is properly aligned with the skull.

The disclosed technology can support the development of high-sensitivity harmonic imaging as well as therapeutic interventions that employ nonlinear acoustics. By augmenting nonlinear wave propagation through engineered layers or by utilizing the nonlinear properties of intervening structures, the disclosed technology can enhance parametric array signal generation for various diagnostic and therapeutic applications.

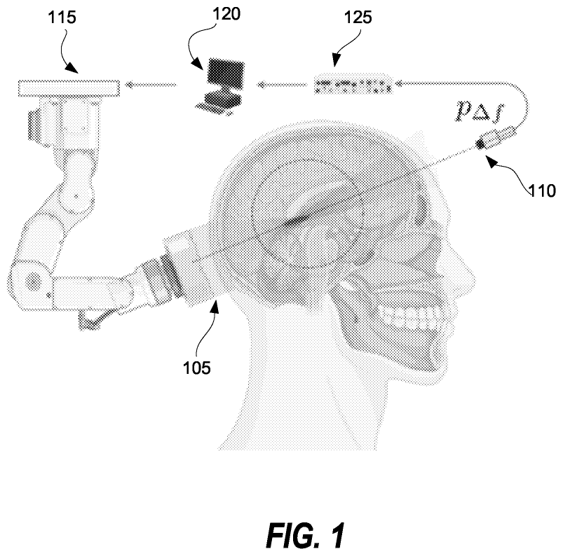

FIG. 1 illustrates a system 100 for focused ultrasound therapy registration. The system 100 can include an ultrasound transducer 105 configured to generate a first acoustic wave at a first frequency and a second acoustic wave at a second frequency. The ultrasound transducer 105 can have an active aperture diameter of 60 mm and can be coupled with a single focusing lens having an F-number of 0.75. In some cases, the system 100 can include a linear amplifier providing 55 dB gain before supplying a bi-frequency signal to the ultrasound transducer 105.

The system 100 can include a holographic lens coupled to the ultrasound transducer 105 and configured to focus acoustic energy toward a target region through a nonlinear medium. The holographic lens can be referred to herein as a phase plate. The holographic lens can be configured to correct for aberrations introduced by the nonlinear medium. In some cases, the nonlinear medium can comprise a skull. The holographic lens can be designed using a heterogeneous angular spectrum approach combined with an ADAM iterative optimizer for topology optimization. The holographic lens can be fabricated using 3D printing with resin-based materials having a compressional wave attenuation of approximately 3 dB/MHz.

The system 100 can include a receiver 110 configured to detect a parametric array signal generated by nonlinear mixing of the first acoustic wave and the second acoustic wave within the nonlinear medium. The parametric array signal can comprise a difference frequency component corresponding to a difference between the first frequency and the second frequency. The receiver 110 can be positioned at a fixed location relative to a patient's head to detect acoustic signals including parametric array signals generated through nonlinear wave interactions within the skull.

The system 100 can include a robotic arm 115 coupled to the ultrasound transducer 105 and configured to adjust a position or orientation of the ultrasound transducer 105 relative to the nonlinear medium based on an alignment status. The robotic arm 115 can provide six degrees of freedom for positioning and alignment of the ultrasound transducer 105 relative to the patient's skull. The robotic arm 115 can receive positioning commands from a controller 120.

The system 100 can include the controller 120 configured to receive the parametric array signal from the receiver 110 and determine the alignment status between the holographic lens and the nonlinear medium based on a characteristic of the parametric array signal. The controller 120 can analyze the parametric array signal characteristics to determine when the holographic lens is aligned with the skull. In some cases, the controller 120 can determine that the holographic lens is aligned with the nonlinear medium when the parametric array signal exhibits a minimum amplitude. The controller 120 can utilize the relationship between skull nonlinearity and aberrations caused by misregistration to determine when the holographic lens is properly aligned with the skull.

The system 100 can include a low pass filter and amplifier 125 for processing the detected signal from the receiver 110. The low pass filter and amplifier 125 can filter and amplify the difference frequency component of the parametric array signal. The processed signal from the low pass filter and amplifier 125 can be transmitted to the controller 120 for analysis. In some cases, the low pass filter and amplifier 125 can provide 40 dB of low-pass filtering to isolate the difference frequency component from the primary frequency components.

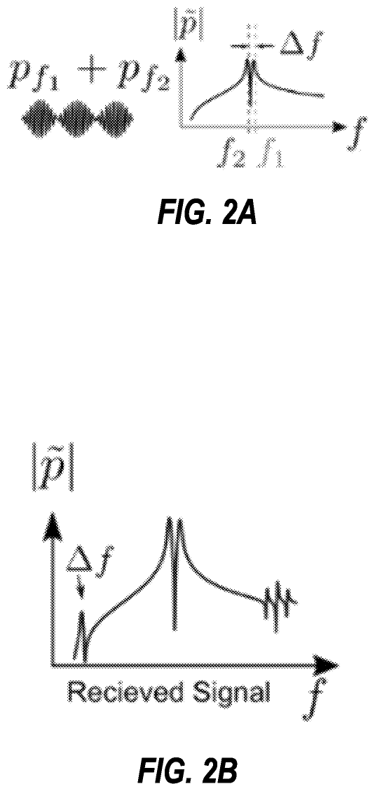

FIGS. 2A-2B illustrate frequency domain representations of acoustic signals associated with the parametric array effect. FIG. 2A depicts a source pressure signal in the frequency domain. The source pressure signal can comprise a bi-frequency signal containing two primary frequencies, denoted as f₁ and f₂. The bi-frequency signal can contain a first frequency f₁ of 0.95 MHz and a second frequency f₂ of 1.05 MHz. The frequency spectrum of the source pressure signal displays two distinct peaks at the first frequency f₁ and the second frequency f₂. The frequency difference between the two primary frequencies can be denoted as Δf, where Δf=f₂−f₁. In some cases, the difference frequency Δf can be 100 kHz.

FIG. 2B depicts a received signal in the frequency domain, illustrating the result of nonlinear acoustic propagation through a nonlinear medium. The received signal spectrum displays a low-frequency component at the difference frequency Δf on the left side of the spectrum, representing the difference frequency component generated through the parametric array effect. The received signal spectrum can also display higher frequency components corresponding to the primary frequencies f₁ and f₂ on the right side of the spectrum. The presence of the difference frequency component in the received signal demonstrates the nonlinear mixing of the two primary high-frequency waves during propagation through the nonlinear medium.

As described above with reference to FIG. 1, the system 100 can include a low pass filter and amplifier 125 configured to filter the parametric array signal to isolate the difference frequency component. The low pass filter and amplifier 125 can be configured with a cutoff frequency of 600 kHz. The low pass filter and amplifier 125 can provide 40 dB gain for filtering the measured signals received from the receiver 110. The low pass filter can isolate the difference frequency component from the primary frequency components, allowing the controller 120 to analyze the parametric array signal characteristics for determining alignment status between the holographic lens and the nonlinear medium.

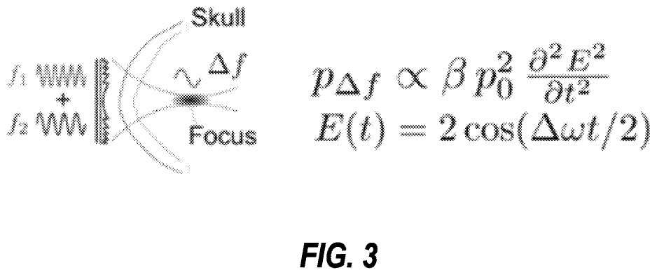

FIG. 3 illustrates nonlinear mixing of two high-frequency acoustic waves through a skull to produce a difference frequency signal. As described above with reference to FIG. 1, an ultrasound transducer coupled to a holographic lens can transmit a first acoustic wave at a first frequency f₁ and a second acoustic wave at a second frequency f₂ toward a nonlinear medium. The nonlinear medium can comprise a skull. A receiver can detect a parametric array signal generated by nonlinear mixing of the first acoustic wave and the second acoustic wave within the nonlinear medium. The parametric array signal can comprise a difference frequency component corresponding to a difference between the first frequency and the second frequency.

In FIG. 3, the two input waveforms at frequencies f₁ and f₂ are combined and propagate toward the skull. Within the skull region, the waves interact nonlinearly, producing a difference frequency signal denoted as Δf. A focal point is indicated within the skull cavity where the acoustic energy converges.

The parametric pressure at the difference frequency p_{Δf} can be proportional to the nonlinearity parameter β, the square of the primary beam pressure

$${p}_{0}^{2} ,$$

and the second partial derivative of the envelope function E² with respect to time squared. This relationship can be expressed as:

The envelope function E(t) can be defined as:

where Δω represents the angular difference frequency corresponding to the difference between the angular frequencies of the first acoustic wave and the second acoustic wave.

The nonlinear medium can comprise a skull having a nonlinearity parameter β of approximately 372, which can be equivalent to a Goldberg number of 4.75. The nonlinearity parameter of the skull can be substantially greater than the nonlinearity parameter of surrounding soft tissue or water, which can have a nonlinearity parameter of approximately 3.5 to 4.5. This contrast in nonlinearity parameters can cause the parametric array signal generation to be dominated by the volume of the skull illuminated by high-intensity ultrasound.

When the two high-frequency waves pass through the skull, the waves mix nonlinearly to generate a low-frequency difference signal that can be detected and used for registration or monitoring purposes. The strength of the parametric array signal can be proportional to the nonlinearity parameter of the medium, the amplitude of the primary beams, and the propagation length. The skull volume can act as a local amplifier for the generation of the difference frequency signal due to the high nonlinearity parameter of the skull bone compared to surrounding tissue.

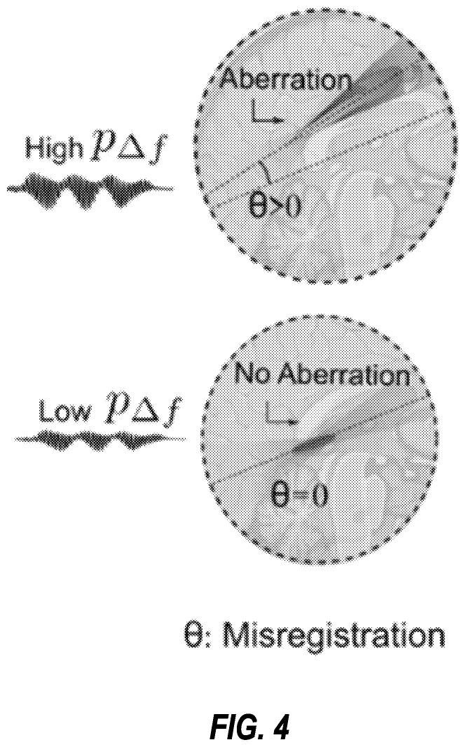

FIG. 4 illustrates a relationship between parametric array signal strength and misregistration in a focused ultrasound system. The misregistration can be characterized by a misregistration angle θ, which represents the rotational offset between the holographic lens and the nonlinear medium. As described above with reference to FIG. 1, the system 100 can include a controller 120 configured to determine an alignment status between the holographic lens and the nonlinear medium based on a characteristic of the parametric array signal.

FIG. 4 depicts two conditions corresponding to different alignment states. In a first condition where the misregistration angle θ is greater than zero, the parametric array signal exhibits a high amplitude. The high parametric array signal corresponds to a state where aberration is present due to misalignment between the holographic lens and the nonlinear medium. A circular inset in FIG. 4 displays a cross-sectional view of brain tissue with an elongated focal region, indicating the presence of aberration due to lens-skull misalignment. The system 100 can be configured to detect skull-induced aberrations that cause axial elongation of the primary focal fields without substantial reduction in peak focal pressure. The axial elongation of the primary focal fields extends the nonlinear interaction distance, which leads to a larger parametric array signal.

In a second condition where the misregistration angle θ equals zero, the parametric array signal exhibits a minimum amplitude. The minimum amplitude parametric array signal corresponds to a state where no aberration is present, indicating proper alignment between the holographic lens and the nonlinear medium. A circular inset in FIG. 4 displays a cross-sectional view of brain tissue with a compact focal region, indicating that the holographic lens is properly registered with the skull. When the holographic lens is aligned with the nonlinear medium, the focal region exhibits a compact profile rather than an axially elongated profile.

The controller 120 can be configured to determine that the holographic lens is aligned with the nonlinear medium when the parametric array signal exhibits a minimum amplitude. Analyzing the parametric array signal can comprise detecting a drop in amplitude of the difference frequency component, wherein the drop in amplitude corresponds to alignment of the holographic lens with the nonlinear medium. The controller 120 can be configured to determine alignment by detecting a drop in parametric pressure of at least 20% when skull rotation is zero degrees. The relationship between parametric array signal amplitude and misregistration angle provides a feedback mechanism for alignment verification, where the parametric array signal drops to a minimum value when the holographic lens is properly registered with the skull.

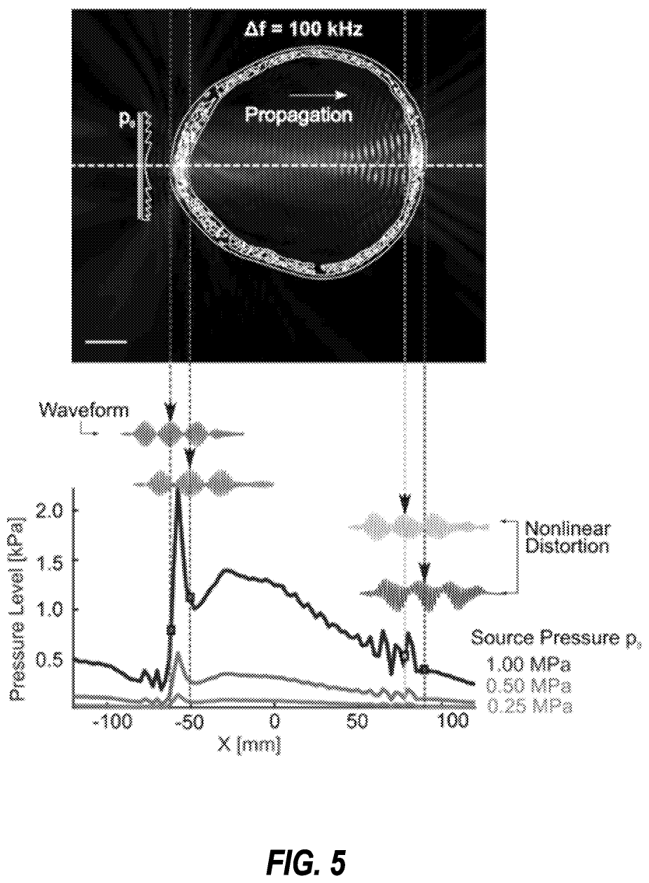

FIG. 5 illustrates nonlinear sound propagation and parametric array signal generation through a skull structure. The upper portion of FIG. 5 depicts a two-dimensional acoustic simulation demonstrating generation of a 100 kHz parametric field. An oval-shaped skull cross-section is shown with a textured boundary representing the skull bone, and a propagation direction is indicated by an arrow pointing to the right. A source pressure po is shown on the left side of the skull structure. The difference frequency is labeled as Δf=100 kHz.

The lower portion of FIG. 5 presents a graph showing pressure level in kilopascals on the vertical axis versus position X in millimeters on the horizontal axis. Three curves are displayed corresponding to different source pressures of 1.00 MPa, 0.50 MPa, and 0.25 MPa. The graph demonstrates waveform characteristics at different positions along the propagation axis, with waveform insets shown at approximately −50 mm and 100 mm positions. The waveforms at the −50 mm position show primary wave characteristics, while the waveforms at the 100 mm position demonstrate nonlinear distortion. The pressure traces show a peak near the −50 mm position corresponding to the skull location, with the highest source pressure of 1.00 MPa producing the largest pressure response reaching approximately 2.0 kPa. A dashed horizontal line extends across the figure connecting the skull simulation to the corresponding positions on the pressure graph below.

As described above with reference to FIGS. 2A-2B, the first frequency and the second frequency can be in a range of 0.9 MHz to 1.1 MHz. In some cases, the first frequency f₁ can be 0.95 MHz and the second frequency f₂ can be 1.05 MHz. The difference frequency component can be in a range of 50 kHz to 200 kHz. In some cases, the difference frequency Δf can be 100 kHz.

The waveforms in FIG. 5 show increasing nonlinear distortion as primary waves attenuate due to skull-induced losses. The parametric array signal can be immediately evident after the primary beams pass through the skull. The system can be configured to operate with primary beams ranging from 0.25 MPa to 1 MPa for safe exposure during parametric array signal generation. The pressure traces along the axial direction demonstrate that higher source pressures produce correspondingly larger parametric array signals.

The system can operate with pulse lengths of 30 to 40 cycles of a primary 1 MHz pulse, resulting in a total pulse duration of 30 to 40 microseconds. The short tone bursts can avoid standing wave artifacts during parametric array signal generation. The pulse parameters can be configured to generate parametric array signals without crossing thermal and mechanical safety thresholds.

The two-dimensional acoustic simulation in FIG. 5 demonstrates that the parametric field is generated within the skull cavity and subsequently transmitted through the skull cavity. The skull volume can act as a local amplifier for the generation of the difference frequency signal due to the high nonlinearity parameter of the skull bone compared to surrounding tissue. The accumulation of nonlinearities over extended propagation distances, combined with the substantially higher attenuation of the primary MHz-range fields, can result in the parametric array signal being the dominant low-frequency component in the received signal. The 100 kHz parametric array signal can pass through the skull with minimal loss compared to the primary frequency components, as attenuation is proportional to frequency.

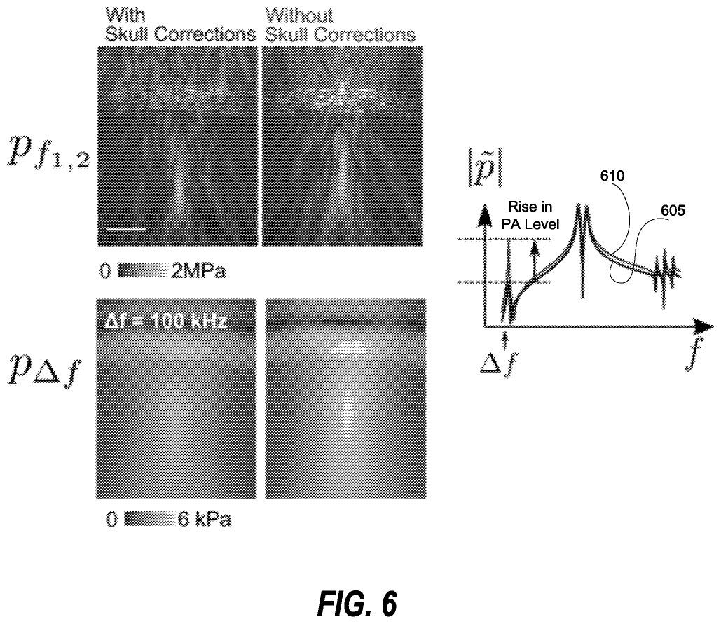

FIG. 6 illustrates a comparison of parametric array signal generation with and without skull corrections. As described above with reference to FIG. 1, the nonlinear medium can comprise a skull, and the holographic lens can be configured to correct for aberrations introduced by the skull. FIG. 6 depicts acoustic field images comparing the primary field pressure p_{f}_{_{1,2}} and the parametric field pressure p_{Δf} under two conditions: with skull corrections and without skull corrections.

The left side of FIG. 6 displays four acoustic field images arranged in a two-by-two grid. The top row shows the primary field pressure p_{f}_{_{1,2}} with skull corrections on the left and without skull corrections on the right. The bottom row shows the parametric field pressure p_{Δf} at a difference frequency Δf of 100 kHz, with skull corrections on the left and without skull corrections on the right. The images without skull corrections demonstrate axially elongated primary focal fields compared to the corrected case.

The right side of FIG. 6 depicts a graph plotting the absolute value of pressure against frequency. A curve with skull corrections 605 shows a relatively narrow peak with minimal distortion. A curve without skull corrections 610 exhibits a broader profile with increased nonlinear distortion. The graph indicates a rise in parametric array level corresponding to the difference frequency Δf.

Aberrated beams produce higher parametric array signals because the aberrated beams extend the nonlinear interaction distance within the skull. When the holographic lens is not properly registered with the skull, the primary focal fields become axially elongated without a substantial reduction in peak focal pressure. The axial elongation of the primary focal fields extends the region over which nonlinear wave interactions occur, which leads to a larger parametric array signal. The curve without skull corrections 610 demonstrates this effect, where the broader profile corresponds to the extended nonlinear interaction region.

The system 100 can detect volumetric energy trapping within the high-nonlinearity skull volume. When misalignment is present between the holographic lens and the skull, the trapped energy within the skull volume increases. Integration of the squared primary pressure within the skull bone volume can reveal an increase in trapped acoustic energy of approximately 9% during a 2 degree misalignment. The increase in trapped acoustic energy amplifies the nonlinear source term, which is proportional to the product of the nonlinearity parameter β and the square of the primary pressure magnitude. The volumetric energy trapping within the skull acts as a pump that amplifies the parametric array signal generation.

The aberration-induced signal enhancement relates to alignment detection by providing a measurable difference between aligned and misaligned states. When the holographic lens is properly aligned with the skull, the curve with skull corrections 605 exhibits a narrow peak corresponding to a compact focal region. When the holographic lens is misaligned with the skull, the curve without skull corrections 610 exhibits a broader profile corresponding to an elongated focal region and increased parametric array signal generation. The controller 120 can utilize this relationship to determine the alignment status between the holographic lens and the nonlinear medium, where a drop in the parametric array signal corresponds to proper alignment.

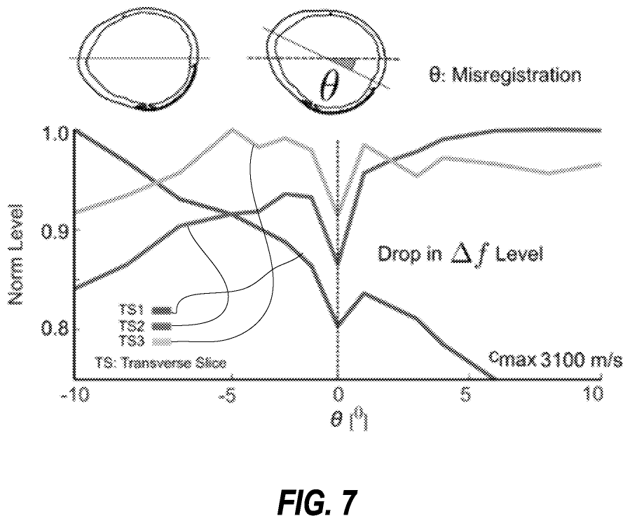

FIG. 7 illustrates a relationship between normalized level and misregistration angle for parametric array signal analysis during skull-lens alignment. The upper portion of FIG. 7 shows two schematic representations of skull cross-sections, with a left schematic showing an aligned configuration and a right schematic showing a rotated configuration with an angle θ indicating the misregistration. A dashed horizontal line connects the two schematics to illustrate a reference alignment axis.

The main graph in FIG. 7 plots normalized level on the vertical axis against the misregistration angle θ in degrees on the horizontal axis. The horizontal axis ranges from negative ten degrees to positive ten degrees. Three curves are displayed corresponding to three different transverse skull slices, labeled as TS1, TS2, and TS3. A vertical dashed line at zero degrees indicates an optimal registration position where θ equals zero.

The graph in FIG. 7 demonstrates a drop in the difference frequency level at the zero-degree position. The drop corresponds to optimal skull-lens alignment. Regardless of which transverse skull slice is used, the parametric pressure exhibits a characteristic minimum at zero misregistration. The drop in the difference frequency level at zero degrees confirms that the parametric array signal can serve as a feedback mechanism for detecting proper alignment between a holographic lens and the skull.

As described above with reference to FIG. 1, the system 100 can include a robotic arm 115 configured to adjust a position or orientation of the ultrasound transducer 105 relative to the nonlinear medium based on an alignment status. Adjusting the position or orientation of the holographic lens can comprise rotating the holographic lens about at least one axis until the parametric array signal reaches a minimum amplitude. The skull can be mounted on a rotating fixture that allows rotation at 1 degree increments around the z-axis for registration measurements.

The notation in FIG. 7 indicates a maximum speed of sound of 3100 meters per second. The maximum speed of sound parameter can be used in simulations to model the acoustic properties of the skull bone. The results shown in FIG. 7 persist across different skull slices, indicating that the parametric array signal drop is a robust metric for alignment detection regardless of variations in skull geometry or acoustic properties among different transverse skull slices.

The curves for TS1, TS2, and TS3 each show a pronounced decrease in normalized level at zero misregistration. The persistence of the parametric pressure drop across different transverse skull slices indicates that aberration induced by misregistration of the holographic lens position relative to skull orientation has a tangible impact on the parametric array signal. The controller 120 can utilize the relationship between the normalized level and the misregistration angle to determine when the holographic lens is properly aligned with the skull, where the parametric array signal drops to a minimum value at zero degrees rotation.

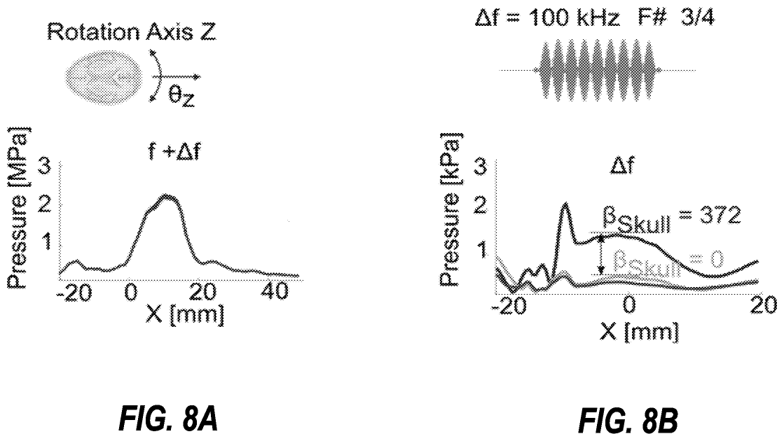

FIGS. 8A-8B illustrate pressure measurements as a function of position comparing the primary frequency and the difference frequency under different skull nonlinearity conditions. FIG. 8A depicts pressure measured in megapascals (MPa) on the vertical axis plotted against X position in millimeters on the horizontal axis. FIG. 8A corresponds to a configuration with a rotation axis Z and an angle θ_{z}, with a frequency offset f+Δf applied. FIG. 8B depicts pressure measured in kilopascals (kPa) on the vertical axis plotted against X position in millimeters on the horizontal axis. FIG. 8B illustrates the difference frequency Δf and includes two curves representing different skull nonlinearity parameters.

As described above with reference to FIG. 3, the nonlinear medium can comprise a skull having a nonlinearity parameter β. In FIGS. 8A-8B, the skull nonlinearity parameter βₛₖᵤₗₗ can have a value of 372 or a value of 0. When the skull nonlinearity parameter βₛₖᵤₗₗ equals 372, the parametric pressure signal exhibits a higher amplitude curve. When the skull nonlinearity parameter βₛₖᵤₗₗ equals 0, the parametric pressure signal exhibits a lower amplitude curve. The operating parameters indicated in FIGS. 8A-8B include a difference frequency Δf of 100 kHz and an F-number of 3/4.

The graphs in FIGS. 8A-8B demonstrate that when skull nonlinearity is present at a value of 372, the parametric pressure signal is enhanced compared to when skull nonlinearity is absent. The primary field at the primary frequency can remain fixed across different levels of skull nonlinearity. The parametric field at the difference frequency can be sensitive to skull nonlinearity. The parametric field can increase when skull nonlinearity is present and can decrease when skull nonlinearity is absent.

The skull nonlinearity can augment nonlinear wave propagation and parametric array signal generation. The nonlinear medium can have a nonlinearity parameter of at least 20. In some cases, the nonlinear layer can have a nonlinearity parameter of at least 20. The B/A nonlinearity parameter can be in two digits for the registration strategy to function. In some cases, the nonlinearity parameter can be at least 20 to 30. The skull can have a nonlinearity parameter β of approximately 372, which can be equivalent to a Goldberg number of 4.75. The nonlinearity parameter of the skull can be substantially greater than the nonlinearity parameter of surrounding soft tissue or water.

The contrast in nonlinearity parameters between the skull and surrounding tissue can cause the parametric array signal generation to be dominated by the volume of the skull illuminated by high-intensity ultrasound. A nonlinear layer positioned in the propagation path of acoustic waves can enhance the generation of harmonics, self-phase demodulation, and other nonlinear acoustic effects. The nonlinear layer can support the development of finite amplitude effects at shorter propagation distances than would occur in the absence of the nonlinear layer. The skull volume can act as a local amplifier for the generation of the difference frequency signal due to the high nonlinearity parameter of the skull bone compared to surrounding tissue.

The results shown in FIGS. 8A-8B indicate that the parametric array signal can serve as a feedback mechanism for alignment detection. The sensitivity of the parametric field to skull nonlinearity can provide a measurable difference between conditions where skull nonlinearity is present and conditions where skull nonlinearity is absent. The controller can utilize the relationship between skull nonlinearity and parametric array signal amplitude to determine the alignment status between the holographic lens and the nonlinear medium.

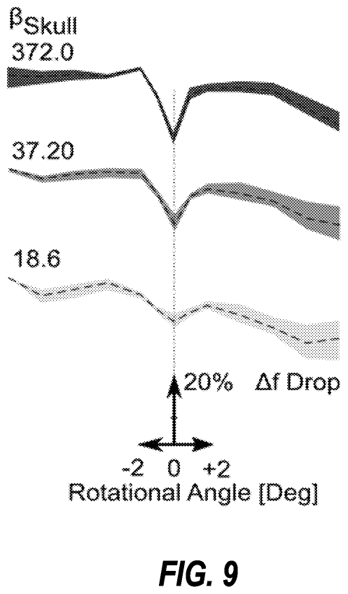

FIG. 9 illustrates a relationship between parametric pressure drop and rotational angle for different skull nonlinearity values. The system can use pseudo-spectral time-domain (PSTD) nonlinear acoustic simulations of a k-Wave toolbox for modeling nonlinear wave propagation through the skull. As described above with reference to FIG. 1, the system 100 can include an ultrasound transducer configured to generate a first acoustic wave at a first frequency and a second acoustic wave at a second frequency. A nonlinear layer can be positioned in a propagation path of the first acoustic wave and the second acoustic wave. The nonlinear layer can be configured to augment generation of a parametric array signal through nonlinear mixing of the first acoustic wave and the second acoustic wave. A receiver can be configured to detect the parametric array signal. The parametric array signal can comprise a difference frequency component corresponding to a difference between the first frequency and the second frequency.

The graph in FIG. 9 depicts a vertical axis representing a twenty percent change in difference frequency drop and a horizontal axis representing rotational angle in degrees ranging from negative two to positive two degrees. Three curves are shown corresponding to different skull nonlinearity parameter values of 372.0, 37.20, and 18.6. The skull nonlinearity parameter values of 372.0, 37.20, and 18.6 can be equivalent to Goldberg numbers of 4.75, 0.475, and 0.237, respectively. Each curve is displayed with a dashed line indicating a mean value and a shaded region representing a variance or uncertainty range.

The curve corresponding to the skull nonlinearity value of 372.0 exhibits the most pronounced drop in parametric pressure at zero degrees rotation, indicating optimal registration between the holographic lens and the skull. The curve corresponding to the skull nonlinearity value of 37.20 shows a moderate drop at zero degrees rotation. The curve corresponding to the skull nonlinearity value of 18.6 displays a relatively flat response with minimal variation across the rotational angle range. A vertical dashed line indicates the zero degree position where the parametric pressure drop is most pronounced for higher nonlinearity values.

The graph in FIG. 9 demonstrates that higher skull nonlinearity leads to greater sensitivity in detecting misregistration between the holographic lens and the skull through changes in the parametric array signal. The reduction in parametric pressure diminishes as skull nonlinearity decreases. When skull misalignment is minimized such that the rotational angle θ equals zero, the drop in the parametric array signal becomes more pronounced for higher skull nonlinearity values. The nonlinear layer can have a nonlinearity parameter greater than a nonlinearity parameter of water. The nonlinearity parameter of water can be approximately 3.5 to 4.5. The skull can have a nonlinearity parameter β of approximately 372, which can be substantially greater than the nonlinearity parameter of water.

The simulations can be performed by rotating a three-dimensional skull model in a transverse plane and performing multiple registration iterations. The three-dimensional nonlinear simulations can produce a more pronounced drop in the parametric array signal at zero misregistration compared to two-dimensional simulations for the same nonlinearity parameters. The ability of nonlinear effects to compound in three-dimensional propagation can contribute to the enhanced sensitivity of the parametric array signal to misregistration.

The results shown in FIG. 9 indicate that the parametric array signal can serve as a feedback mechanism for alignment detection when the nonlinear layer has a nonlinearity parameter of at least 20. The sensitivity of the parametric pressure drop to skull nonlinearity can provide a measurable difference between aligned and misaligned states. The controller can utilize the relationship between skull nonlinearity and parametric pressure drop to determine the alignment status between the holographic lens and the nonlinear medium, where a drop in the parametric array signal corresponds to proper alignment at zero degrees rotation.

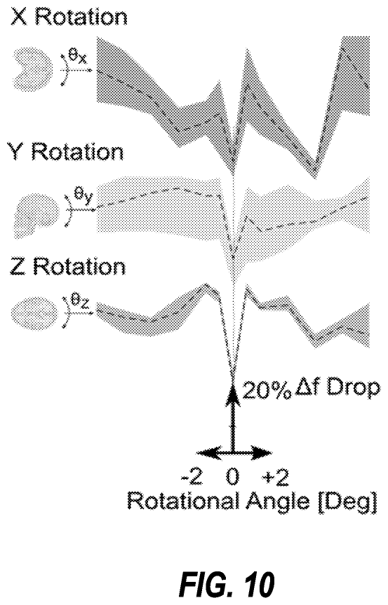

FIG. 10 illustrates variation of parametric pressure with rotational angle for three different axes of rotation. As described above with reference to FIG. 1, the system 100 can include an ultrasound transducer configured to generate a first acoustic wave at a first frequency and a second acoustic wave at a second frequency. A nonlinear layer can be positioned in a propagation path of the first acoustic wave and the second acoustic wave. The nonlinear layer can comprise bone tissue. The nonlinear layer can be configured to augment generation of a parametric array signal through nonlinear mixing of the first acoustic wave and the second acoustic wave.

FIG. 10 depicts three separate plots arranged vertically, each corresponding to rotation about a different axis: X Rotation, Y Rotation, and Z Rotation. Each plot displays a dashed line representing the measured parametric pressure response as a function of rotational angle in degrees. The horizontal axis spans from approximately negative two degrees to positive two degrees. A shaded gray region surrounds each dashed line, indicating a range of variability or uncertainty in the measurements. Small skull diagrams appear to the left of each plot, illustrating the respective rotation axis with angular indicators labeled as θₓ, θ_{y}, and θ_{z} for the X, Y, and Z rotations respectively.

A vertical scale indicator labeled as twenty percent Δf Drop is positioned at the bottom center of FIG. 10, providing a reference for the magnitude of the parametric pressure changes. The twenty percent Δf Drop reference scale indicates the magnitude of the parametric pressure reduction that can be observed when the holographic lens is properly aligned with the nonlinear medium.

The plots in FIG. 10 demonstrate that rotations about each axis produce distinct parametric pressure responses. The Z Rotation plot shows a more pronounced dip near zero degrees compared to the X Rotation and Y Rotation plots. The X Rotation and Y Rotation plots exhibit relatively flatter responses with smaller variations across the angular range. Rotations about the z-axis result in more substantial decreases in parametric pressure compared to rotations about the x and y axes.

The drop in the parametric array signal during alignment can be robust to different axes of rotation. The controller can utilize the relationship between parametric pressure and rotational angle for each axis to determine the alignment status between the holographic lens and the nonlinear medium. The more pronounced dip in parametric pressure for z-axis rotation can provide a stronger feedback signal for alignment detection when the holographic lens is rotated about the z-axis. The system can be configured to monitor parametric pressure changes during rotation about multiple axes to achieve alignment between the holographic lens and the nonlinear medium.

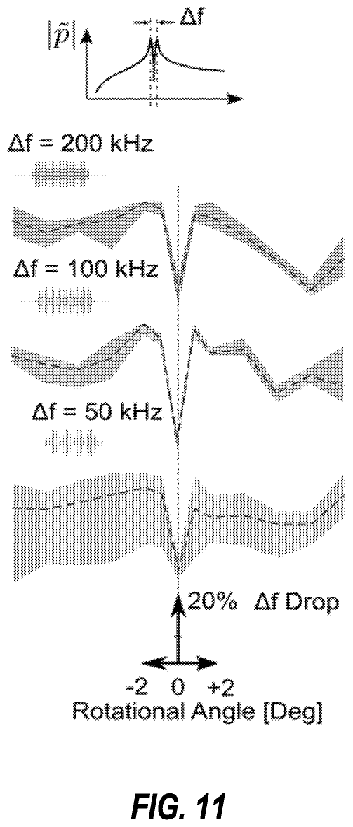

FIG. 11 illustrates an effect of varying difference frequencies on parametric pressure drop as a function of rotational angle. As described above with reference to FIG. 1, the system 100 can include an ultrasound transducer configured to generate a first acoustic wave at a first frequency and a second acoustic wave at a second frequency. A nonlinear layer can be positioned in a propagation path of the first acoustic wave and the second acoustic wave. The nonlinear layer can be configured to augment generation of a parametric array signal through nonlinear mixing of the first acoustic wave and the second acoustic wave. The nonlinear layer can be configured to support generation of harmonics and self-phase demodulation at a shorter propagation distance than would occur in absence of the nonlinear layer.

FIG. 11 depicts three different difference frequency conditions corresponding to Δf values of 200 kHz, 100 kHz, and 50 kHz. Each condition is represented by a dashed line with a shaded region indicating measurement uncertainty or variability. The horizontal axis represents rotational angle in degrees, ranging from negative two degrees to positive two degrees. The vertical axis represents parametric pressure in arbitrary units. At the top of FIG. 11, a small inset diagram shows a frequency spectrum with a peak, illustrating the definition of Δf as the bandwidth or difference frequency parameter.

For each difference frequency condition, a small waveform illustration is provided showing the corresponding signal characteristics. Higher difference frequencies display more compressed waveforms, and lower difference frequencies display more extended waveforms. All three curves demonstrate a pronounced dip in parametric pressure at zero degrees rotational angle, indicating alignment between the holographic lens and the nonlinear medium.

Lower difference frequencies lead to relatively modest drops in parametric pressure. The curve for Δf equals 50 kHz shows a drop of approximately twenty percent at the zero degree position. The shaded uncertainty regions are narrower for the 200 kHz condition and progressively wider for the 100 kHz and 50 kHz conditions. The results indicate that the downshift ratio is a parameter for selecting the difference frequency in registration applications. The downshift ratio can be defined as the ratio of the primary frequency to the difference frequency f/Δf. Larger downshift ratios can yield smaller parametric array signals due to lower nonlinear interaction efficiency.

Higher difference frequency values can enhance parametric pressure generation up to the point that skull attenuation becomes significant. Generating higher difference frequencies such as 200 kHz can require a wider separation between the primary frequencies. The wider separation between primary frequencies can be limited by the bandwidth of piezoelectric transducers. A larger separation between primary frequencies can reduce primary pressure amplitudes.

The difference frequency can be selected based on a trade-off between nonlinear conversion efficiency and practical limitations of transducer bandwidth. The operational frequency window for the difference frequency can be in the range of 75 kHz to 125 kHz for hydrocephalus monitoring applications. In the 75 kHz to 125 kHz range, the parametric conversion efficiency can be sufficient to generate a measurable signal. The beam can maintain effective collimation to interact with target structures within the skull cavity in the 75 kHz to 125 kHz range.

At low difference frequencies such as 50 kHz or below, the nonlinear conversion efficiency can drop as expected from the theory of parametric acoustic arrays. Additionally, larger acoustic wavelengths associated with lower difference frequencies can lead to diffraction, resulting in a diffuse beam that can reduce spatial sensitivity. At higher difference frequencies such as 100 kHz or above, the beam can maintain collimation for improved spatial sensitivity.

The controller can utilize the relationship between difference frequency and parametric pressure drop to select an appropriate difference frequency for alignment detection. The system can be configured to operate with a difference frequency in the range of 75 kHz to 125 kHz to achieve a balance between parametric conversion efficiency and beam collimation. The parametric array signal drop at zero degrees rotation can provide a feedback mechanism for alignment detection across the range of difference frequencies from 50 kHz to 200 kHz.

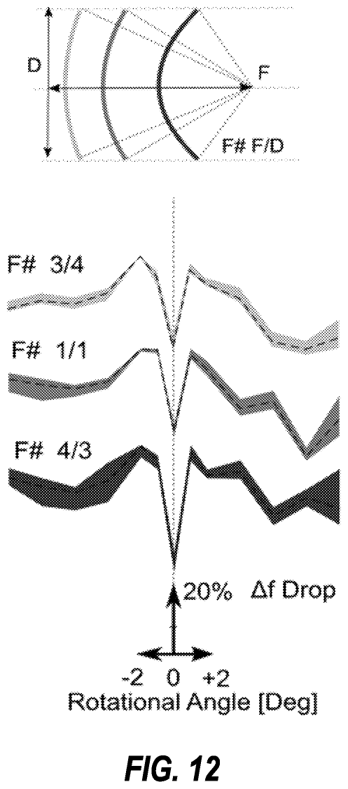

FIG. 12 illustrates a relationship between F-number and parametric pressure drop sensitivity for different focusing configurations. As described above with reference to FIG. 1, the system 100 can include an ultrasound transducer configured to generate a first acoustic wave at a first frequency and a second acoustic wave at a second frequency. A holographic lens can be coupled to the ultrasound transducer and configured to focus the first acoustic wave and the second acoustic wave toward a target region. The F-number can be defined as the ratio of focal length F to aperture diameter D.

The upper portion of FIG. 12 shows a schematic representation of a curved acoustic element with diameter D and focal length F. Three curved lines of varying shades represent different curvatures corresponding to different F-numbers. Dotted lines extend from the curved element to indicate the focal region. The F-number determines the focusing characteristics of the acoustic beam, where smaller F-numbers correspond to tighter focusing and larger F-numbers correspond to weaker focusing.

The lower portion of FIG. 12 displays three waveform plots corresponding to three different F-number values: F #3/4, F #1/1, and F #4/3. Each plot shows the variation of a measured signal as a function of rotational angle in degrees, ranging from approximately −2 degrees to +2 degrees. The waveforms exhibit a characteristic dip at zero degrees rotation, with the magnitude of the dip indicated as a 20% change in difference frequency. The shaded regions around each waveform represent measurement uncertainty or variability. The dashed lines within each waveform indicate a reference or mean value.

The plots in FIG. 12 demonstrate that smaller F-number values result in more pronounced reductions in parametric pressure at zero rotation angle. The F #3/4 configuration, which corresponds to tighter focusing, exhibits a larger dip in parametric pressure at zero degrees compared to the F #1/1 and F #4/3 configurations. The F #4/3 configuration, which corresponds to weaker focusing, shows a less pronounced dip in parametric pressure at zero degrees rotation.

The relationship between F-number and parametric pressure drop sensitivity can be attributed to the propagation distance over which nonlinear effects accumulate. A tightly focused beam with a smaller F-number can accumulate less nonlinearity over the short propagation distance to the focal region. When aberration is present due to misregistration between the holographic lens and the nonlinear medium, the aberration has a higher differential impact on the parametric array signal for tightly focused beams. The shorter propagation distance associated with smaller F-numbers can result in a more pronounced change in parametric pressure when the alignment status changes between aligned and misaligned states.

Conversely, a weakly focused beam with a larger F-number can accumulate nonlinearity over a longer propagation distance. The extended propagation distance can reduce the differential impact of aberration on the parametric array signal. The parametric pressure drop at zero degrees rotation can be less pronounced for larger F-number configurations because the nonlinear interaction region is distributed over a longer axial distance.

The focal depth can influence the sensitivity of the alignment detection method. Reducing the F-number while maintaining a constant aperture can produce a progressively more tightly focused beam with a shorter focal depth. The shorter focal depth can increase the sensitivity of the parametric array signal to misregistration between the holographic lens and the nonlinear medium. The system can be configured with an F-number selected based on the desired sensitivity for alignment detection.

A vertical dashed line in FIG. 12 connects the focal point in the upper schematic to the zero-degree position in the lower plots, indicating the correspondence between alignment of the holographic lens and the minimum in parametric signal. The controller can utilize the relationship between F-number and parametric pressure drop to determine the alignment status between the holographic lens and the nonlinear medium. The system can be configured to operate with an F-number of 3/4 to achieve enhanced sensitivity for alignment detection, or with a larger F-number when reduced sensitivity is acceptable for a particular application.

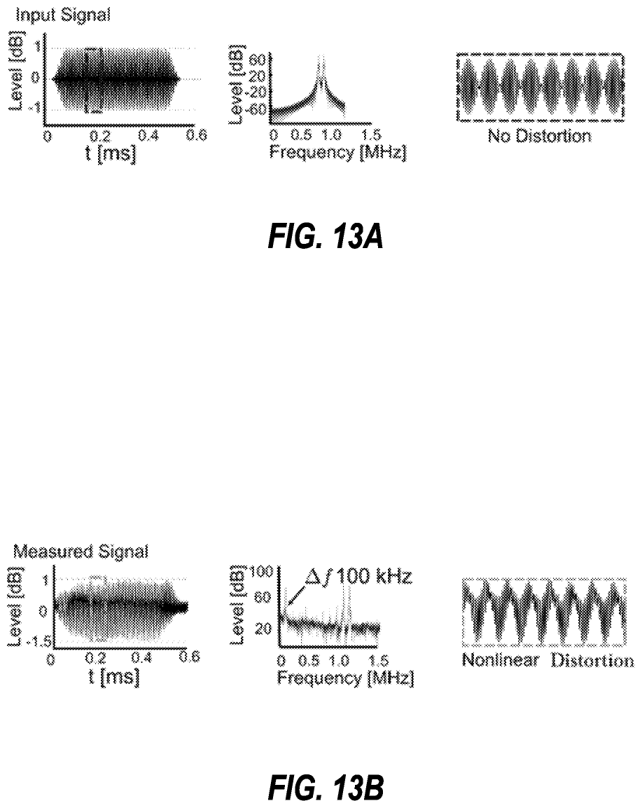

FIGS. 13A-13B illustrate a comparison between an input signal and a measured signal in both time and frequency domains, demonstrating the presence of nonlinear distortion resulting from propagation through a nonlinear medium. As described above with reference to FIG. 1, the system 100 can include an ultrasound transducer configured to generate a first acoustic wave at a first frequency and a second acoustic wave at a second frequency. The ultrasound transducer can be excited with a bi-frequency signal containing primary frequencies of 0.95 MHz and 1.05 MHz to produce a 100 kHz difference frequency.

FIG. 13A depicts the input signal characteristics in both time and frequency domains. The time-domain representation displays level in decibels (dB) versus time in milliseconds, ranging from 0 to 0.6 ms. The frequency-domain representation displays level in dB versus frequency in MHz, ranging from 0 to 1.5 MHz. The frequency spectrum of the input signal exhibits two peaks around 1.0 MHz, corresponding to the two primary frequency components f₁ and f₂. A zoomed-in section of the time-domain waveform in FIG. 13A is labeled as having no distortion. The bi-frequency signal demonstrates a clean waveform with two primary frequency components and no visible distortion in the time-domain envelope. The input signal can be amplified by a linear amplifier providing 55 dB gain before supplying the bi-frequency signal to the ultrasound transducer.

FIG. 13B depicts the measured signal characteristics in both time and frequency domains after propagation through the nonlinear medium. The time-domain representation displays level in dB versus time in milliseconds, ranging from 0 to 0.6 ms. The frequency-domain representation displays level in dB versus frequency in MHz, ranging from 0 to 1.5 MHz. The frequency spectrum of the measured signal exhibits a difference frequency component at 100 kHz, in addition to the primary frequency components. A zoomed-in section of the time-domain waveform in FIG. 13B is labeled as exhibiting nonlinear distortion. The measured signal demonstrates the generation of additional frequency components including a low-frequency difference component at 100 kHz, along with visible nonlinear distortion in the time-domain waveform envelope characterized by amplitude modulation and asymmetric peaks.

The measured signal in FIG. 13B can be obtained using a hydrophone and after 600 kHz low pass filtering with 40 dB gain. The low pass filter can isolate the difference frequency component from the primary frequency components. The presence of the difference frequency component in the measured signal demonstrates the nonlinear mixing of the two primary high-frequency waves during propagation through the nonlinear medium.

The comparison between FIG. 13A and FIG. 13B illustrates the parametric array effect, where the bi-frequency signal with no distortion is transformed into a measured signal exhibiting nonlinear distortion after propagation through the nonlinear medium. The zoomed-in sections in FIGS. 13A-13B highlight the contrast between the undistorted input waveform and the distorted measured waveform. The nonlinear distortion visible in the measured signal corresponds to the generation of the parametric array signal through nonlinear wave interactions within the nonlinear medium.

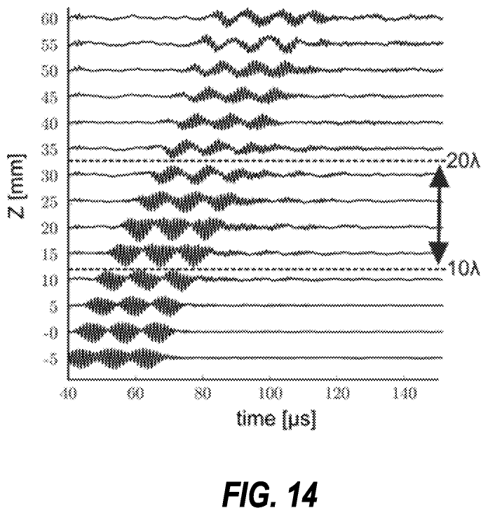

FIG. 14 illustrates progressive nonlinear distortion of a measured signal at different axial positions. FIG. 14 depicts a stacked waterfall plot where the vertical axis represents the axial position Z in millimeters and the horizontal axis represents time in microseconds. Multiple waveform traces are displayed at different axial positions, with each trace offset vertically according to the corresponding Z position. The waveforms show oscillatory patterns with varying amplitudes along the time axis.

Two horizontal dashed lines are indicated on the plot in FIG. 14, one at approximately fifteen millimeters labeled as ten wavelengths and another at approximately thirty-three millimeters labeled as twenty wavelengths. The two horizontal dashed lines define a measurement window region between ten wavelengths and twenty wavelengths. The measurement window can be used for characterizing the self-demodulation effect that occurs as the acoustic signal propagates away from the focal position.

The waveforms at lower Z positions near the focal region exhibit higher amplitude oscillations with more pronounced nonlinear distortion characteristics. The waveforms at higher Z positions show reduced amplitude and different temporal profiles. The progressive change in waveform shape along the axial direction demonstrates the self-demodulation effect occurring as the acoustic signal propagates away from the focal position. Axial line scans can confirm that a 0.2 MPa peak-to-peak primary field at the focus produces increasing nonlinear distortion along the axis beyond the focal position.

A receiver can be configured to detect the parametric array signal at a position between 10 and 20 wavelengths away from the focus. The measurement window between 10 and 20 wavelengths can minimize pseudo-sound effects that can appear when the receiver is subject to strong primary pressure fields. Pseudo-sound effects can arise from the nonlinear transfer function of the receiver and can bias measurements. By positioning the receiver within the measurement window between 10 and 20 wavelengths from the focus, the system can isolate true parametric generation from pseudo-sound artifacts.

The stacked waterfall plot in FIG. 14 demonstrates that the parametric array signal can be detected at positions beyond the focal region where the primary pressure fields have attenuated. The primary pressure can decrease with increasing distance from the focus following an inverse-square relationship. Pseudo-sound depends on the primary pressure amplitude and can drop following a similar trend. The parametric array signal can remain relatively flat across the measurement window, indicating that the measured signal at those distances corresponds to true parametric generation that accumulates over a longer propagation distance rather than hydrophone nonlinearity.

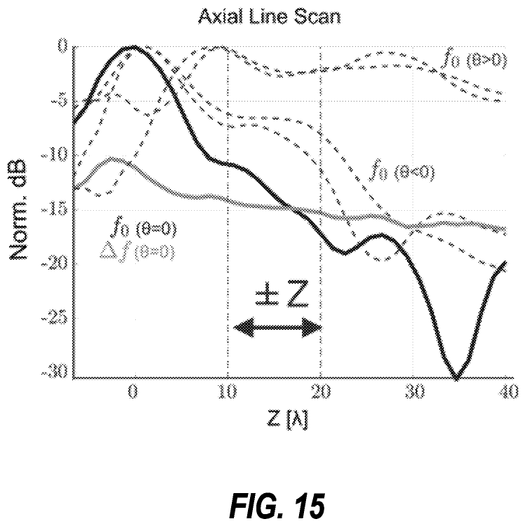

FIG. 15 illustrates an axial line scan showing normalized amplitude versus axial position in wavelengths. The graph in FIG. 15 depicts normalized amplitude in decibels (Norm. dB) on the vertical axis versus axial position Z measured in wavelengths on the horizontal axis. The vertical axis ranges from approximately 0 dB at the top to −30 dB at the bottom, while the horizontal axis extends from 0 to 40 wavelengths.

As described above with reference to FIG. 1, the receiver can comprise a hydrophone configured to perform axial line scans to characterize the parametric array signal at different distances from the skull. The hydrophone can be mounted on a three-axis positioning system to enable scanning along the axial direction. The skull cavity can be immersed in degassed water to provide a standardized acoustic environment and to minimize the risk of pseudo-sound artifacts due to hydrophone nonlinearity.

The graph in FIG. 15 contains multiple curves representing different measurement conditions. A solid black curve represents the measurement at the center frequency f₀ when the rotation angle θ equals zero. A solid gray curve represents the measurement at the difference frequency Δf when the rotation angle θ equals zero. Two dashed curves represent measurements at the center frequency f₀ for positive rotation angles (θ>0) and negative rotation angles (θ<0).

The solid black curve for θ equals zero shows a peak near Z equals 0 wavelengths and then decreases to approximately −30 dB near Z equals 35 wavelengths before rising slightly. The dashed curves for non-zero rotation angles maintain relatively higher amplitude values across the axial range compared to the θ equals zero condition. The line scans for the primary frequency reveal broadening of the axial beamwidth for both positive (θ>0) and negative (θ<0) registration errors. The broadening of the axial beam profiles indicates that misregistration between the holographic lens and the nonlinear medium leads to axial elongation of the focal region.

Two vertical dashed lines are positioned at approximately Z equals 10 wavelengths and Z equals 20 wavelengths, defining a measurement window labeled ±Z between the two vertical dashed lines. The measurement window between 10 and 20 wavelengths can be used to minimize pseudo-sound effects that can appear when the hydrophone is subject to strong primary pressure fields. Pseudo-sound effects can arise from the nonlinear transfer function of the hydrophone and can bias measurements. By positioning the hydrophone within the measurement window between 10 and 20 wavelengths from the focus, the system can isolate true parametric generation from pseudo-sound artifacts.

The gray curve representing the difference frequency signal at θ equals zero shows lower amplitude values throughout the measurement range compared to the primary frequency curves. The difference frequency signal can remain relatively flat across the measurement window, indicating that the measured signal at those distances corresponds to true parametric generation that accumulates over a longer propagation distance rather than hydrophone nonlinearity.

The receiver can be configured with a broad receiver window of 20 mm positioned in the far-field to capture the bulk of forward-propagating energy flux and average out local geometric steering artifacts caused by skull curvature. The broad receiver window can act as a spatial integrator. The spatial integration provided by the broad receiver window can capture the forward-propagating energy flux and can average out local geometric steering artifacts. The receiver can be positioned at a fixed location while the holographic lens orientation is adjusted to find the minimum parametric array signal.

The measurements aggregated across z-axis positions can confirm the pronounced drop in parametric pressure when the skull rotation is zero. The normalization in FIG. 15 can be performed across the different rotational angles (θ) for measurements taken at specific axial positions. The axial line scan data can support the relationship between misregistration and axial beam profile broadening, where proper alignment of the holographic lens with the nonlinear medium corresponds to a compact focal region and a minimum in the parametric array signal.

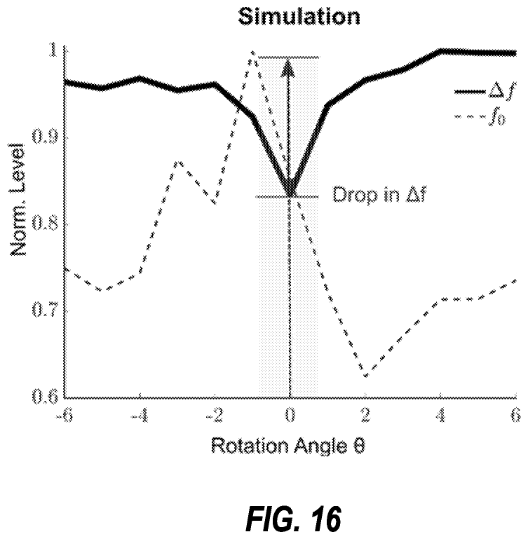

FIG. 16 illustrates simulation results showing a relationship between normalized level and rotation angle for alignment detection. The system can use PSTD nonlinear acoustic simulations of a k-Wave toolbox for modeling nonlinear wave propagation through the skull. The k-Wave toolbox can solve nonlinear coupled wave equations using skull and tissue nonlinearity parameters.

The graph in FIG. 16 depicts normalized level on the vertical axis plotted against rotation angle θ on the horizontal axis. The horizontal axis ranges from negative six degrees to positive six degrees. A first curve represented by a solid line corresponds to the difference frequency Δf. A second curve represented by a dashed line corresponds to the primary frequency f₀.

The first curve corresponding to the difference frequency Δf shows relatively stable values near 0.95 to 1.0 across rotation angles away from zero degrees, with a drop occurring near zero degrees rotation. The second curve corresponding to the primary frequency f₀ exhibits more variation across the rotation angle range, showing values that fluctuate between approximately 0.65 and 0.95, with a pronounced dip occurring near zero degrees where the normalized level drops to approximately 0.65.

A shaded region near the zero degree rotation angle highlights the area where both curves show minimum values. The shaded region indicates the condition corresponding to alignment between the holographic lens and the nonlinear medium. The drop in the difference frequency Δf at zero degrees rotation is indicated by a vertical arrow and horizontal line marking the decrease in the parametric array signal at the zero rotation position.

The simulation results in FIG. 16 demonstrate that the parametric array signal can serve as a feedback mechanism for alignment detection. The drop in the difference frequency signal at zero degrees rotation corresponds to proper alignment between the holographic lens and the nonlinear medium. The controller can utilize the relationship between normalized level and rotation angle to determine the alignment status, where a drop in the parametric array signal corresponds to proper alignment at zero degrees rotation.

The system can exploit mode-converted shear waves within the skull that amplify the parametric array effect. The efficiency of nonlinear parametric interaction can scale inversely with the cube of wave velocity c⁻³. Shear waves in bone can have a velocity cₛ of approximately 1400 m/s, which can be slower than longitudinal waves having a velocity cₗ of approximately 2800 m/s. The ratio of parametric generation efficiency between shear waves and longitudinal waves can be expressed as:

The inverse cube scaling with wave velocity can result in an approximately eight-fold generation efficiency increase when longitudinal waves are mode-converted into shear waves within the skull. Angular misregistration can trigger volumetric energy trapping and shear mode conversion through which misaligned primary acoustic energy can be trapped within the diploë layer and can be converted into slow-moving shear waves. The trapped energy can act as a volumetric pump that amplifies the parametric array signal generation.

The receiver can be configured to detect the parametric array signal from a position on an acoustically coupled head surface. The parametric array interaction volume can have a wavelength λ_{Δf} of approximately 15 mm, which can be larger than the skull thickness. A source smaller than the wavelength λ_{Δf} can radiate as a monopole. The monopole-like behavior of the parametric array interaction volume can allow the receiver to detect the alignment-induced energy dip from any acoustically coupled position on the head surface. The receiver can require no phase alignment with the transmitter due to the monopole-like radiation pattern of the parametric array source.

The three-dimensional nonlinear simulations can produce a more pronounced drop in the parametric array signal at zero misregistration compared to two-dimensional simulations for the same nonlinearity parameters. The ability of nonlinear effects to compound in three-dimensional propagation can contribute to the enhanced sensitivity of the parametric array signal to misregistration. The simulations can be performed by rotating a three-dimensional skull model in a transverse plane and performing multiple registration iterations.

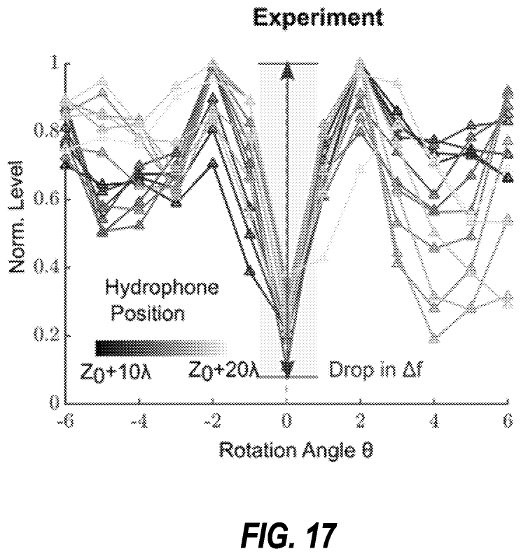

FIG. 17 illustrates experimental variation of parametric pressure as a function of rotation angle θ. As described above with reference to FIG. 1, the system 100 can include an ultrasound transducer configured to generate a first acoustic wave at a first frequency and a second acoustic wave at a second frequency. A receiver can be configured to detect a parametric array signal generated by nonlinear mixing of the first acoustic wave and the second acoustic wave within a nonlinear medium. The parametric array signal can comprise a difference frequency component corresponding to a difference between the first frequency and the second frequency.

The graph in FIG. 17 plots normalized level on the vertical axis against rotation angle θ on the horizontal axis. The horizontal axis ranges from negative six degrees to positive six degrees. Multiple data series are plotted using triangle markers connected by lines. The shading of the markers and lines varies from dark to light to indicate different hydrophone positions ranging from Z₀+10λ to Z₀+20λ, as shown in a gradient legend labeled “Hydrophone Position” in the lower left portion of the graph.

The graph in FIG. 17 demonstrates that the normalized level values are generally higher at rotation angles away from zero, with values typically ranging between approximately 0.6 and 1.0 at the outer angles. A pronounced drop in the difference frequency Δf is observed near the zero rotation angle, indicated by a shaded vertical band centered at θ=0 with an arrow pointing downward labeled “Drop in Δf.” The drop corresponds to a decrease in normalized level to approximately 0.4 or below for most data series.

The experimental data in FIG. 17 demonstrates that the parametric pressure decreases when the skull rotation approaches zero, indicating effective registration of the holographic lens with the skull. The measurements aggregated across z-axis positions confirm the pronounced drop in parametric pressure when the skull rotation is zero. The normalization in FIG. 17 can be performed across the different rotational angles θ for measurements taken at specific axial positions. The receiver can be positioned at a fixed location while the holographic lens orientation is adjusted to find the minimum parametric array signal.

The multiple data series corresponding to different hydrophone positions from Z₀+10λ to Z₀+20λ demonstrate distance-independent operation of the parametric array signal detection. The primary pressure can decrease with increasing distance from the focus following an inverse-square relationship. Pseudo-sound effects can depend on the primary pressure amplitude and can drop following a similar trend. The parametric array signal can remain relatively flat across the measurement window, indicating that the measured signal at those distances corresponds to true parametric generation that accumulates over a longer propagation distance rather than hydrophone nonlinearity. The system can be configured such that the distance between the transducer and the nonlinear medium does not affect the parametric array signal generation due to the high nonlinearity of the medium.

The parametric array interaction volume can have a wavelength λ_{Δf} of approximately 15 mm, which can be larger than the skull thickness. A source smaller than the wavelength Δ_{Δf} can radiate as a monopole. The monopole-like behavior of the parametric array interaction volume can allow the receiver to detect the alignment-induced energy dip from any acoustically coupled position on the head surface. The receiver can require no phase alignment with the transmitter due to the monopole-like radiation pattern of the parametric array source.

As described above with reference to FIG. 1, the controller can be configured to analyze the parametric array signal to determine an alignment status between the holographic lens and the nonlinear medium. The controller can utilize the relationship between normalized level and rotation angle to determine the alignment status, where a drop in the parametric array signal corresponds to proper alignment at zero degrees rotation. The system can be configured to adjust a position or orientation of the holographic lens relative to the nonlinear medium based on the alignment status.

The system can be configured to be tolerant to lateral probe misalignment of plus or minus 5 mm while maintaining diagnostic contrast for hydrocephalus monitoring applications. The tolerance to lateral misalignment can support the development of wearable sensor designs for continuous monitoring applications. The parametric array signal suppression as a diagnostic contrast can remain stable across a wide operational frequency band of 75 kHz to 125 kHz.

In some cases, a nonlinear layer can be positioned in a propagation path of the first acoustic wave and the second acoustic wave. The nonlinear layer can be configured to augment generation of the parametric array signal through nonlinear mixing of the first acoustic wave and the second acoustic wave. In some cases, the nonlinear layer can comprise materials having nonlinearity greater than water, such as alcohol or ethylene glycol, which have nonlinearity approximately twice that of water. However, for applications requiring more pronounced parametric array signal generation, materials with nonlinearity parameters of at least 20 may be preferred. In some cases, the nonlinear layer can comprise 3D-printed material having structural properties similar to skull, including a calcium matrix structure. In some cases, the nonlinear layer can comprise femur bone, tibia, or other porous bone structures having high nonlinearity comparable to skull. The nonlinear layer can have a nonlinearity parameter of at least 20.

The experimental results in FIG. 17 support the relationship between misregistration and parametric array signal amplitude, where proper alignment of the holographic lens with the nonlinear medium corresponds to a minimum in the parametric array signal. The persistence of the parametric pressure drop across different hydrophone positions indicates that the registration metric can be robust to hydrophone placement and aperture size variations. The characteristic dip in parametric array signal at zero degrees rotation can remain consistent and detectable despite variations in hydrophone placement across the measurement aperture.

The disclosed technology can be applied to additional applications including hydrocephalus monitoring. Hydrocephalus is a condition characterized by ventricular expansion within the brain, which can serve as a proxy for changes in intracranial pressure. The parametric array signal can be used to non-invasively monitor ventricular expansion by detecting shifts in the brain's effective acoustic nonlinearity in response to relative changes in ventricular size.

The disclosed technology can also be applied to imaging applications in body regions beyond the skull. In some cases, a nonlinear layer can be positioned on or near other body parts such as the abdomen for difference frequency imaging applications. A nonlinear layer patch can be placed on the skin surface to augment parametric array signal generation, where the nonlinear layer enhances the generation of the difference frequency signal for improved imaging contrast. The nonlinear layer can comprise bone tissue or other highly nonlinear materials positioned between the ultrasound transducer and the imaging target. By positioning a nonlinear layer on the abdomen or other body parts, the disclosed technology can support difference frequency imaging in regions where natural highly nonlinear structures are not present.

The system can be configured to detect ventricular expansion in hydrocephalus by monitoring changes in the parametric array signal. Expanding ventricles can replace higher-nonlinearity brain tissue with lower-nonlinearity cerebrospinal fluid (CSF) in the interaction zone. The displacement of brain tissue by CSF can result in lower total nonlinear acoustic energy at the difference frequency. The system can be configured such that a 10% drop in signal amplitude indicates pathological ventricular expansion. The 10% signal attenuation can be above the detection threshold of a hydrophone and can provide a diagnostic margin for reliable differentiation between normal and hydrocephalus states.

The separation between normal and hydrocephalus states can enable threshold-based classification. A parametric array signal below a baseline threshold can indicate pathological ventricular expansion. The threshold-based classification can operate without complex image reconstruction or expert operator interpretation. The controller can be configured to compare the measured parametric array signal to a baseline value and to generate a clinical alert when the measured signal drops by more than a threshold percentage relative to the baseline.

The system can operate with a pulse repetition frequency of 1-2 Hz for hydrocephalus monitoring applications. The pulse repetition frequency can correspond to a desired sensor refresh rate. The pulse parameters can be configured to generate parametric array signals without crossing thermal and mechanical safety thresholds. The pulse length for the bi-frequency excitation sequence can be constrained to avoid auditory neuromodulation or other adverse artifacts caused by long, low-frequency pulses. The system can operate with short bursts consisting of 30 to 40 cycles of a primary 1 MHz pulse, resulting in a total pulse duration of 30 to 40 microseconds.

The diagnostic contrast can be defined as the separation between the parametric array signal amplitude for normal brain tissue and the parametric array signal amplitude for hydrocephalus states. The diagnostic contrast can remain stable at approximately 10-15% across the operational frequency band. The diagnostic contrast can rely on changes in the medium's effective nonlinearity parameter as CSF replaces brain tissue in the interaction zone.

The receiver can act as a spatial integrator due to a broad receiver window positioned in the far field. The broad receiver window can capture the bulk of forward-propagating energy flux and can average out local geometric steering artifacts caused by skull curvature. The hydrocephalic brain can produce less nonlinear signal due to CSF replacement, which can lead to lower integrated signal at the difference frequency. The lower integrated signal can create a persistent diagnostic margin for reliable differentiation between normal and hydrocephalus states.

The system can be configured to detect ventricular shrinkage during successful shunt treatment. Monitoring relative changes in the parametric pressure outside the skull cavity can enable tracking of ventricular expansion during hydrocephalus progression and ventricular shrinkage during treatment. The parametric array-based method can serve as a continuous, bulk proxy measurement to track changes in the effective nonlinearity parameter as CSF volume changes within the ventricles.

The parametric array-based monitoring approach can provide a non-invasive surrogate for tracking ventricular volume after removal of an external ventricular drain. The tolerance to lateral misalignment of plus or minus 5 mm can support the development of wearable sensor designs for continuous monitoring applications. A patient or caregiver applying a wearable sensor based on general anatomical landmarks can have a high probability of obtaining a valid diagnosis due to the built-in tolerance to positioning error.

The disclosed technology can be further understood according to the following clauses:

Clause 1: A system for nonlinear ultrasound imaging, comprising: an ultrasound transducer configured to generate a first acoustic wave at a first frequency and a second acoustic wave at a second frequency, wherein the ultrasound transducer is positionable to transmit the first acoustic wave and the second acoustic wave through a nonlinear layer, and wherein the nonlinear layer augments generation of a parametric array signal through nonlinear mixing of the first acoustic wave and the second acoustic wave; a receiver configured to detect the parametric array signal, wherein the parametric array signal comprises a difference frequency component corresponding to a difference between the first frequency and the second frequency; and a controller configured to process the parametric array signal to generate imaging data based on the difference frequency component.

Clause 2: The system of clause 1, wherein the nonlinear layer has a nonlinearity parameter greater than a nonlinearity parameter of water.

Clause 3: The system of clause 1, wherein the nonlinear layer has a nonlinearity parameter of at least 20.

Clause 4: The system of clause 1, wherein the nonlinear layer comprises bone tissue.

Clause 5: The system of clause 1, wherein the nonlinear layer is configured to support generation of harmonics and self-phase demodulation at a shorter propagation distance than would occur in absence of the nonlinear layer.

Clause 6: The system of clause 1, wherein the first frequency and the second frequency are in a range of 0.9 MHz to 1.1 MHz.

Clause 7: The system of clause 1, wherein the difference frequency component is in a range of 50 kHz to 200 kHz.

Clause 8: The system of clause 1, further comprising a holographic lens coupled to the ultrasound transducer and configured to focus the first acoustic wave and the second acoustic wave toward a target region.

Clause 9: A method for augmenting nonlinear acoustic wave propagation, comprising: transmitting, from an ultrasound transducer, a first acoustic wave at a first frequency and a second acoustic wave at a second frequency through a nonlinear layer, wherein the nonlinear layer generates a parametric array signal through nonlinear mixing of the first acoustic wave and the second acoustic wave, and wherein the parametric array signal comprises a difference frequency component corresponding to a difference between the first frequency and the second frequency; and detecting, with a receiver, the parametric array signal.

Clause 10: The method of clause 9, wherein the nonlinear layer has a nonlinearity parameter greater than a nonlinearity parameter of water.

Clause 11: The method of clause 9, wherein the nonlinear layer has a nonlinearity parameter of at least 20.

Clause 12: The method of clause 9, wherein the nonlinear layer comprises bone tissue.

Clause 13: The method of clause 9, wherein the nonlinear layer is configured to support generation of harmonics and self-phase demodulation at a shorter propagation distance than would occur in absence of the nonlinear layer.

Clause 14: The method of clause 9, further comprising processing the parametric array signal to generate imaging data based on the difference frequency component.

Clause 15: A system for enhancing nonlinear acoustic wave propagation, comprising: an ultrasound transducer configured to generate a first acoustic wave at a first frequency and a second acoustic wave at a second frequency, wherein the ultrasound transducer is positionable to transmit the first acoustic wave and the second acoustic wave through a nonlinear layer, and wherein the nonlinear layer augments generation of a parametric array signal through nonlinear mixing of the first acoustic wave and the second acoustic wave; and a receiver configured to detect the parametric array signal, wherein the parametric array signal comprises a difference frequency component corresponding to a difference between the first frequency and the second frequency.

Clause 16: The system of clause 15, wherein the nonlinear layer has a nonlinearity parameter greater than a nonlinearity parameter of water.

Clause 17: The system of clause 15, wherein the nonlinear layer comprises bone tissue.

Clause 18: The system of clause 15, wherein the nonlinear layer is configured to support generation of harmonics and self-phase demodulation at a shorter propagation distance than would occur in absence of the nonlinear layer.

Clause 19: The system of clause 15, further comprising a holographic lens coupled to the ultrasound transducer and configured to focus the first acoustic wave and the second acoustic wave toward a target region.

Clause 20: The system of clause 15, wherein the nonlinear layer has a nonlinearity parameter of at least 20.

A number of implementations have been described. Nevertheless, it will be understood that various modifications may be made without departing from the spirit and scope of the disclosure. Accordingly, other implementations are within the scope of the following claims.

1. A system for nonlinear ultrasound imaging, comprising:
an ultrasound transducer configured to generate a first acoustic wave at a first frequency and a second acoustic wave at a second frequency, wherein the ultrasound transducer is configured to transmit the first acoustic wave and the second acoustic wave through a nonlinear layer, and wherein the nonlinear layer augments generation of a parametric array signal through nonlinear mixing of the first acoustic wave and the second acoustic wave;
a receiver configured to detect the parametric array signal, wherein the parametric array signal comprises a difference frequency component corresponding to a difference between the first frequency and the second frequency; and
a controller configured to process the parametric array signal to generate imaging data based on the difference frequency component.

2. The system of claim 1, wherein the nonlinear layer has a nonlinearity parameter greater than a nonlinearity parameter of water.

3. The system of claim 1, wherein the nonlinear layer has a nonlinearity parameter of at least 20.

4. The system of claim 1, wherein the nonlinear layer comprises bone tissue.

5. The system of claim 1, wherein the nonlinear layer is configured to support generation of harmonics and self-phase demodulation at a shorter propagation distance than would occur in absence of the nonlinear layer.

6. The system of claim 1, wherein the first frequency and the second frequency are in a range of 0.9 MHz to 1.1 MHz.

7. The system of claim 1, wherein the difference frequency component is in a range of 50 kHz to 200 kHz.

8. The system of claim 1, further comprising a holographic lens coupled to the ultrasound transducer and configured to focus the first acoustic wave and the second acoustic wave toward a target region.

9. A method for augmenting nonlinear acoustic wave propagation, comprising:
transmitting, from an ultrasound transducer, a first acoustic wave at a first frequency and a second acoustic wave at a second frequency through a nonlinear layer, wherein the nonlinear layer generates a parametric array signal through nonlinear mixing of the first acoustic wave and the second acoustic wave, and wherein the parametric array signal comprises a difference frequency component corresponding to a difference between the first frequency and the second frequency; and
detecting, with a receiver, the parametric array signal.

10. The method of claim 9, wherein the nonlinear layer has a nonlinearity parameter greater than a nonlinearity parameter of water.

11. The method of claim 9, wherein the nonlinear layer has a nonlinearity parameter of at least 20.

12. The method of claim 9, wherein the nonlinear layer comprises bone tissue.

13. The method of claim 9, wherein the nonlinear layer is configured to support generation of harmonics and self-phase demodulation at a shorter propagation distance than would occur in absence of the nonlinear layer.

14. The method of claim 9, further comprising processing the parametric array signal to generate imaging data based on the difference frequency component.

15. A system for enhancing nonlinear acoustic wave propagation, comprising:
an ultrasound transducer configured to generate a first acoustic wave at a first frequency and a second acoustic wave at a second frequency, wherein the ultrasound transducer is configured to transmit the first acoustic wave and the second acoustic wave through a nonlinear layer, and wherein the nonlinear layer augments generation of a parametric array signal through nonlinear mixing of the first acoustic wave and the second acoustic wave; and
a receiver configured to detect the parametric array signal, wherein the parametric array signal comprises a difference frequency component corresponding to a difference between the first frequency and the second frequency.

16. The system of claim 15, wherein the nonlinear layer has a nonlinearity parameter greater than a nonlinearity parameter of water.

17. The system of claim 15, wherein the nonlinear layer comprises bone tissue.

18. The system of claim 15, wherein the nonlinear layer is configured to support generation of harmonics and self-phase demodulation at a shorter propagation distance than would occur in absence of the nonlinear layer.

19. The system of claim 15, further comprising a holographic lens coupled to the ultrasound transducer and configured to focus the first acoustic wave and the second acoustic wave toward a target region.

20. The system of claim 15, wherein the nonlinear layer has a nonlinearity parameter of at least 20.
